(19) United States
(12) Patent Application Publication
(10) Pub. No.: US 2014/0319081 A1
(43) Pub. Date: Oct. 30, 2014

(54) SYSTEMS AND METHODS OF MANUFACTURING AUTOLOGOUS SERUM PRODUCTS

(71) Applicant: William J. Davey, San Diego, CA (US)

(72) Inventor: William J. Davey, San Diego, CA (US)

(73) Assignee: San Diego Blood Bank, San Diego, CA (US)

(21) Appl. No.: 13/872,760

(22) Filed: Apr. 29, 2013

(51) Int. Cl. A61M 1/34

(52) U.S. Cl. CPC A61M 1/342
USPC 210/806; 210/96.2; 210/93

(57) ABSTRACT

Methods, systems and kits for production of autologous blood products are provided. Such methods, systems and kits comprise a blood collection set, a serum dilution set, and an aliquot set. The blood collection set includes a collection container and a serum container. The serum dilution set includes a serum measurement device, a serum dilution bag, and a mixing device enabling serum to be mixed with balanced saline solution in a defined dilution ratio. The aliquot set includes at least one filter and aliquot tubing having periodic segment length markings.

### FIELD

The present disclosure relates to systems, methods, and kits for production of autologous serum blood products such as autologous serum eye drops.

### BACKGROUND

Many individuals suffer from severe dryness of the eye, resulting in redness, itching and pain. Many synthetic eye drops are commercially available, and treatment with these drops helps some patients. However, synthetic drops are not effective for many patients. A recently-developed alternative to synthetic eye drops is autologous serum eye drops made from a patient's own blood. These natural drops contain ingredients known to speed healing and increase lubrication of the surface of the eye.

Because the raw material for autologous serum eye drops is the patient's own blood, producing these drops is a complex process involving collection, processing, and dilution of blood serum. Moreover, the process has to comply with certain regulatory requirements. Although some of the components exist for blood collection, to date there is no complete commercial system for making autologous eye drops efficiently and in large scale quantities. Existing systems for collecting blood for autologous blood products involve glass vials, caps, and connections between individual vials to be used during the filling process. However, such systems are inefficient and do not allow for variable dilutions as may be prescribed.

Therefore, there exists a need for a method, system and kit for producing autologous serum eye drops. More particularly, there is a need for a system of producing autologous serum eye drops that includes all supplies and materials for collecting, processing, diluting and dispensing diluted serum product. There is also a need for methods of producing autologous serum eye drops that allow for variable dilutions. Finally, there is a need for a method, system and kit that facilitates efficient and scalable production of autologous serum eye drops.

### SUMMARY

The present disclosure, in its many embodiments, alleviates to a great extent the disadvantages of known blood collection and blood serum production systems by providing methods, systems and kits for production of autologous blood products in which several distinct subsystems having unique features for collecting blood, diluting and filtering serum and aliquotting the diluted serum are provided. The present disclosure provides significant advantages such as facilitating efficient and scalable production of autologous serum eye drops.

Exemplary embodiments include a system of making autologous blood products, comprising a blood collection assembly, a dilution assembly fluidly connected to the blood collection assembly, and an aliquot assembly fluidly connected to the dilution assembly. The blood collection assembly includes a collection container and a serum container. The dilution assembly includes a serum measurement device, a serum dilution bag, and a mixing system enabling serum to be mixed with balanced saline solution in a defined dilution ratio. The aliquot assembly includes at least one filter and aliquot tubing having periodic segment length markings.

In exemplary embodiments, the system further comprises a sealing device to seal each segment of aliquot tubing. The blood collection assembly may further include a needle and a needle guard. The serum container may be a 150 mL bag, and the collection container may be a 150 mL bag. The mixing device may be a syringe. In exemplary embodiments, the at least one filter comprises a materials filter and a sterilization filter, and the materials filter is a 40 micron pre-filter and the sterilization filter is a 0.2 micron filter.

In exemplary embodiments, the mixing system comprises a branched dilution tube including a balanced saline solution input line, a serum container input line, a first clamp to control flow of serum, a second clamp to control flow of balanced saline solution, and a third clamp to control flow of serum-saline solution into the serum dilution bag. Each segment length marking of the aliquot tubing may contain a unique identifier. The aliquot tubing may also define at least one air vent allowing air in the tubing to exit as the tubing is filled with serum-saline solution.

Exemplary embodiments include methods of making autologous blood products. Exemplary methods may comprise the steps of collecting blood from a patient and allowing the blood to clot, separating blood serum and collecting the serum in a container, mixing the serum with balanced saline solution in a defined dilution ratio, filtering the serum-saline solution, directing the serum-saline solution into aliquot tubing, and producing a plurality of aliquots of serum-saline solution by sealing the aliquot tubing at periodic length segments. Such methods may further comprise freezing each aliquot of serum-saline solution. In exemplary embodiments, the filtering step comprises filtering of materials and sterilization filtering. Exemplary methods may further comprise using beads to clot the collected blood. Such methods may also comprise marking each periodic length segment with a unique identifier.

Exemplary embodiments include autologous blood products production kits. Such a kit comprises a blood collection set, a serum dilution set fluidly connected to the blood collection set, and an aliquot set fluidly connected to the serum dilution set. The blood collection set includes a collection container and a serum container. The serum dilution set includes a serum measurement device, a serum dilution bag, and a mixing device enabling serum to be mixed with balanced saline solution in a defined dilution ratio. The aliquot set includes a materials filter, a sterilization filter, and aliquot tubing having periodic segment length markings.

In exemplary embodiments, the kit's mixing system comprises a branched dilution tube including a balanced saline solution input line, a serum container input line, a first clamp to control flow of serum, a second clamp to control flow of balanced saline solution, and a third clamp to control flow of serum-saline solution into the serum dilution bag. Such kits may further comprise a sealing device to seal each segment of aliquot tubing. The blood collection assembly may also include a needle and a needle guard. Each segment length marking of the aliquot tubing may contain a unique identifier.

Accordingly, it is seen that methods, systems and kits are provided which allow efficient and scalable production of autologous serum blood products such as autologous serum eye drops. These and other features of the present invention will be appreciated from review of the following detailed description of the invention, along with the accompanying figures in which like reference numbers refer to like parts throughout.

### BRIEF DESCRIPTION OF THE DRAWINGS

The foregoing and other objects of the disclosure will be apparent upon consideration of the following detailed description, taken in conjunction with the accompanying drawings, in which:

FIG. 1 is a process flow diagram of an exemplary embodiment of a method of making autologous blood products in accordance with the present disclosure;

FIG. 2 is a perspective view of an exemplary embodiment of a blood collection assembly in accordance with the present disclosure;

FIG. 3 is a perspective view of an exemplary embodiment of a dilution assembly in accordance with the present disclosure;

FIG. 4 is a perspective view of an exemplary embodiment of an aliquot assembly in accordance with the present disclosure;

FIG. 5 is a perspective view of an exemplary embodiment of segmented aliquot tubing in accordance with the present disclosure;

FIG. 6 is a perspective view of an exemplary embodiment of separated and bundled aliquot tubing segments in accordance with the present disclosure;

FIG. 7 is a perspective view of an exemplary embodiment of an aliquot tubing segment in accordance with the present disclosure;

FIG. 8 is a perspective view of an exemplary embodiment of an aliquot tubing segment in accordance with the present disclosure;

FIG. 9 is a perspective view of an exemplary embodiment of a materials filter in accordance with the present disclosure; and

FIG. 10 is a perspective view of an exemplary embodiment of an autologous blood products production system or kit in accordance with the present disclosure.

### DETAILED DESCRIPTION

In the following paragraphs, embodiments will be described in detail by way of example with reference to the accompanying drawings, which are not drawn to scale, and the illustrated components are not necessarily drawn proportionately to one another. Throughout this description, the embodiments and examples shown should be considered as exemplars, rather than as limitations of the present disclosure. As used herein, the “present disclosure” refers to any one of the embodiments described herein, and any equivalents. Furthermore, reference to various aspects of the disclosure throughout this document does not mean that all claimed embodiments or methods must include the referenced aspects. Reference to temperature, pressure, density and other parameters should be considered as representative and illustrative of the capabilities of exemplary embodiments, and embodiments can operate with a wide variety of such parameters. It should be noted that the figures do not show every piece of equipment, nor the pressures, temperatures and flow rates of the various streams.

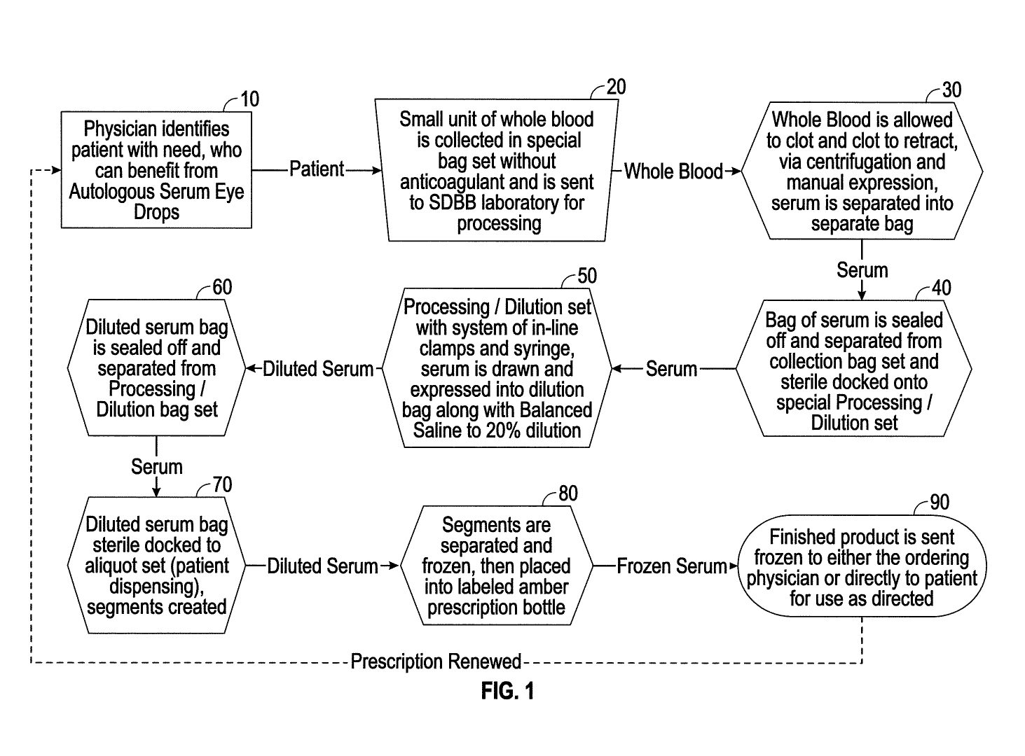

Exemplary embodiments of a system and method of producing autologous blood products will be described in general terms with reference to FIG. 1. Typically, autologous serum eye drops are made using the blood serum, the clear part of the blood, which is diluted with saline to create an eye drop solution. Once a physician identifies 10 a patient who has a need for autologous serum eye drops, the process may begin. The first step 20 is that a small unit of whole blood is collected from the patient. As discussed in more detail herein, a collection set includes a special collection container without anticoagulant. The whole blood is allowed to clot 30, and the clot allowed to retract. The serum is then separated by centrifugation and manual expression and directed to a serum container, which is sealed and separated from the collection assembly and sterile docked 40 onto a special dilution assembly. Next, the serum is drawn and expressed into a serum dilution bag and diluted 50 with balanced saline. The serum dilution bag is then sealed and separated 60 from the dilution assembly and sterile docked 70 to an aliquot assembly, which facilitates creation of segmented aliquots. Finally, the segments are separated and frozen 80. The segmented aliquots of autologous eye drops may be bottled in labeled amber prescription bottles and sent 90 either to the physician or directly to the patient.

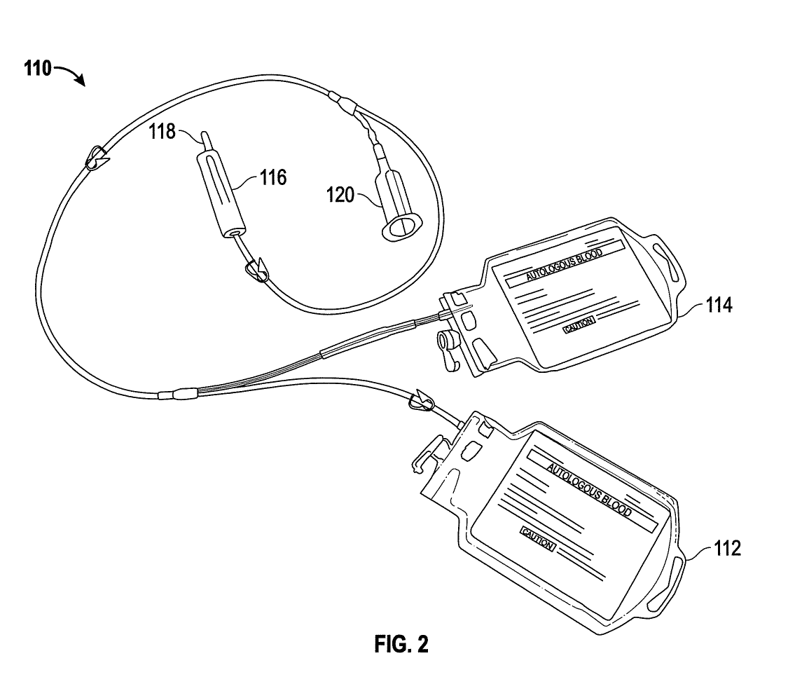

Turning to FIG. 2, an exemplary embodiment of a blood collection assembly or set 110 used to collect whole blood from a patient will be described. A blood collection assembly 110 includes a collection container 112, which may be any type of container suitable for holding blood. In exemplary embodiments, the collection container 112 is a collection bag having a volume capacity of up to about 450 mL, and in some instances, the bag volume is 150 mL. The collection bag 112 has no anticoagulant so the collected blood is not prevented from clotting, resulting in the blood plasma being separated from the serum.

The blood collection assembly 110 also includes a serum container 114 for collecting the serum from the collected blood. More particularly, the serum container 114 is used to express off the serum from the clotted red blood cells. In exemplary embodiments, the serum container 114 is a satellite bag having a volume capacity of up to about 450 mL, and in some instances, the bag volume is 150 mL. A network of tubing and valves connects the collection container 112 to the serum container 114, and also extends to a needle 116 and needle guard 118 for drawing blood from a patient. A vacutainer adapter 120 may also be included in the blood collection assembly 110 for collecting sample tubes of a patients blood.

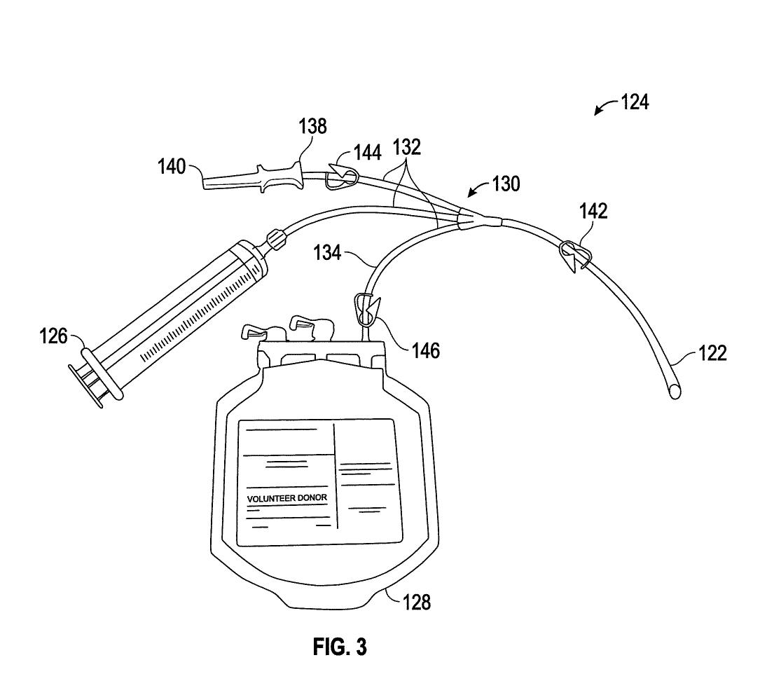

A sterile serum input line of tubing 122 extends from the serum container 114 and provides a fluid connection between the blood collection assembly 110 and the dilution assembly 124, which allows the collected serum to be mixed with balanced saline solution in a defined ratio. With reference to FIG. 3, exemplary embodiments of a dilution assembly or set 124 comprise serum measurement device 126, which could be an integral syringe or any other device suitable for drawing up measured amounts of serum and balanced saline solution. The measurement device 126 is used to measure the serum and balanced saline solution and dispense them into serum dilution bag 128. The serum dilution bag 128 may have a volume capacity of up to about 450 mL, and in some instances, the bag volume is 150 mL. The serum measurement device may have a volume capacity of up to about 250 mL and in exemplary embodiments is 60 mL.

The dilution assembly 124 also includes a mixing system 130 to enable the serum to mixed with balanced saline solution in a defined and adjustable ratio. An exemplary ratio would be about 20% dilution, but other dilutions in the range of about 10% to about 50% could be used. An exemplary embodiment of a mixing system 130 comprises a branched dilution tube network 132 fluidly connected to the serum input line 122. The branched dilution tube network 132 includes a serum dilution bag line 134 fluidly connected to the serum dilution bag 128, a serum measurement device line 136 fluidly connected to the serum measurement device 126, and a balanced saline solution line 138 having a spike 140 at its distal end for asceptic connection to a source of balanced saline solution.

Balanced saline solution flows from a balanced saline solution source (not shown) through balanced saline solution line 138, and serum flows from the serum container 114 of the blood collection assembly 110 through the serum input line 122. The serum measurement device 126 draws up measured amounts of serum and balanced saline solution through the serum measurement device line 136. A first clamp 142 controls the flow of serum through the serum input line 122. A second clamp 144 may be located on the balanced saline solution line 138 and may be used to control the flow of balanced saline solution. A third clamp 146 may be located on the serum dilution bag line 134 to control the flow of serum and saline solution dispensed from the serum measurement device 126 to the serum dilution bag 128. It should be noted that, with the exception of the saline spike, the dilution assembly 124 is a closed system.

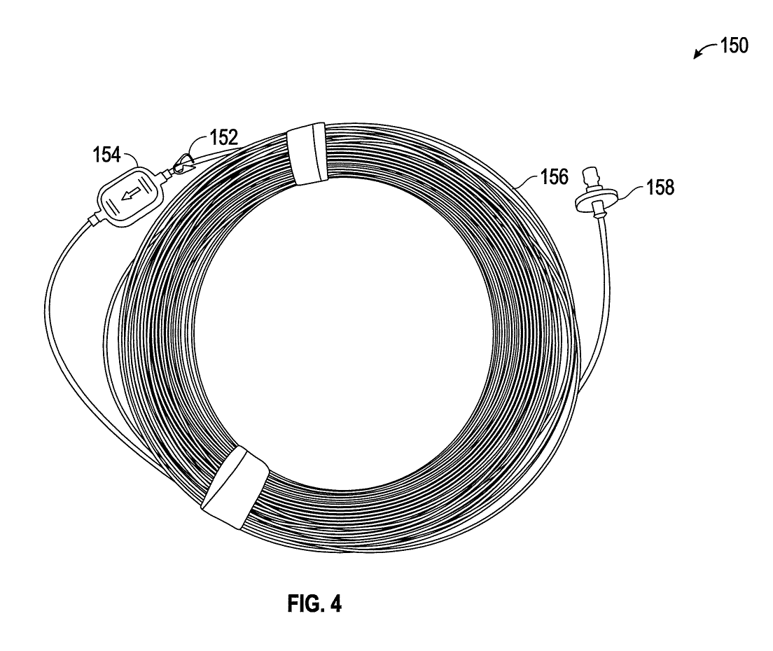

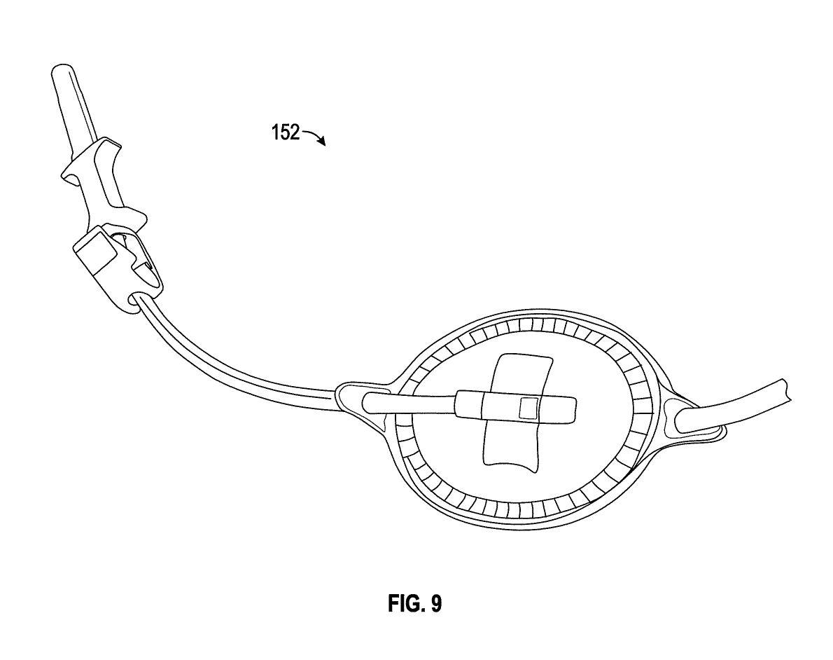

Exemplary embodiments of an aliquot assembly and serum solution aliquots or segments will now be described with reference to FIGS. 4-8. An aliquot assembly or set 150 includes a materials filter 152 (shown in detail in FIG. 9), a sterilization filter 154, and about 1,000 inches of aliquot tubing 156, which are “sterile docked” together via any blood bank sterile connection device currently used for production of blood components. The materials filter 152 is located at a proximal end of the aliquot tubing 156 and acts as a pre-filter to catch and remove any fibrin strands or other clotted material from the serum-saline solution. A range of filter sizes could be utilized for the materials filter 152, such as 20-100 microns, with exemplary embodiments being about 40 microns. Distal to the materials filter 152 is a sterilization filter 154, which makes the diluted serum-saline solution sterile. The sterilization filter 154 filter material is significantly smaller than that of the materials filter 12 and would be in the range of about 0.1-1.0 microns, with exemplary embodiments being about 0.2 microns. The aliquot tubing 156 may have an air vent or valve 158 located at or near its distal end to allow air in the aliquot tubing 156 to exit as the tubing fills with final serum solution while maintaining the sterility of the solution.

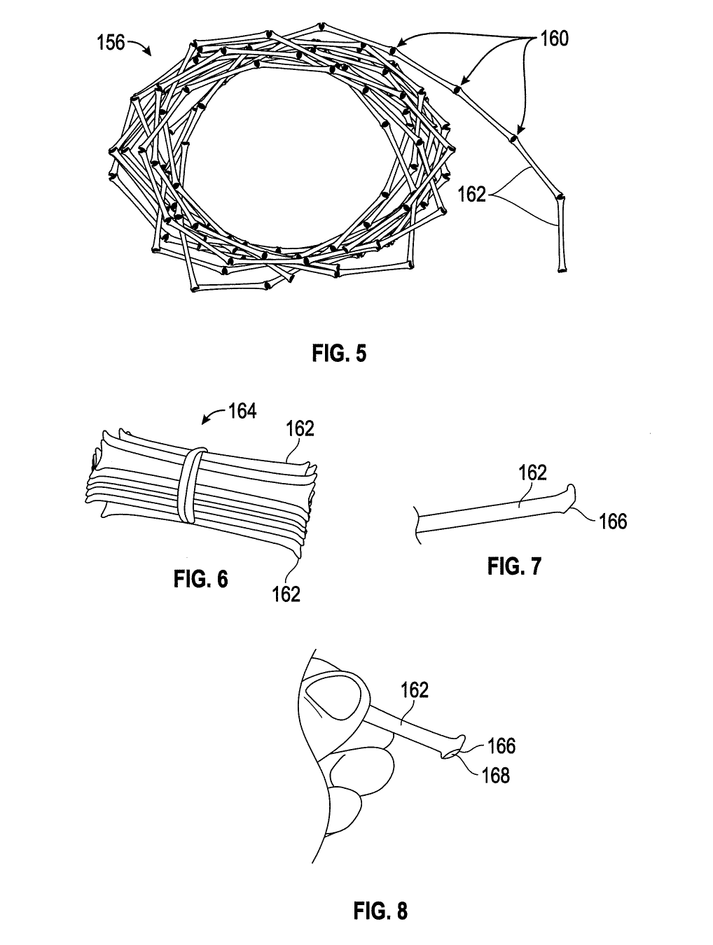

As best seen in FIG. 5, exemplary embodiments of aliquot tubing 156 have periodic segment length markings 160. The markings and resulting segments can be any desired length, including 3 and ¼ inches or 1 and ½ inch lengths, which are most suitable for autologous eye drops. The segment length markings 160 facilitate filling of equal volume aliquots of final serum solution. In exemplary embodiments, each segment is labeled with a unique identifier. More particularly, each marking would be a unique identifier such as a number. The aliquot tubing 156 can be filled with solution then the tubing can be sealed off and the serum solution sealed in short segments 162. As shown in FIG. 6, once separated the segmented aliquots 162 can be arranged in a bundle 164 for ease of transport and storage. The segmented aliquots 162 can be flash frozen at suitable temperature for maintaining freshness and sterility of the autologous eye drops. An appropriate temperature would be about −50° C. As best seen in FIGS. 7 and 8, after thawing, a patient can clip a corner 166 of the segmented aliquot 162 to create an opening 168 suitable for administration of the drops into the eye.

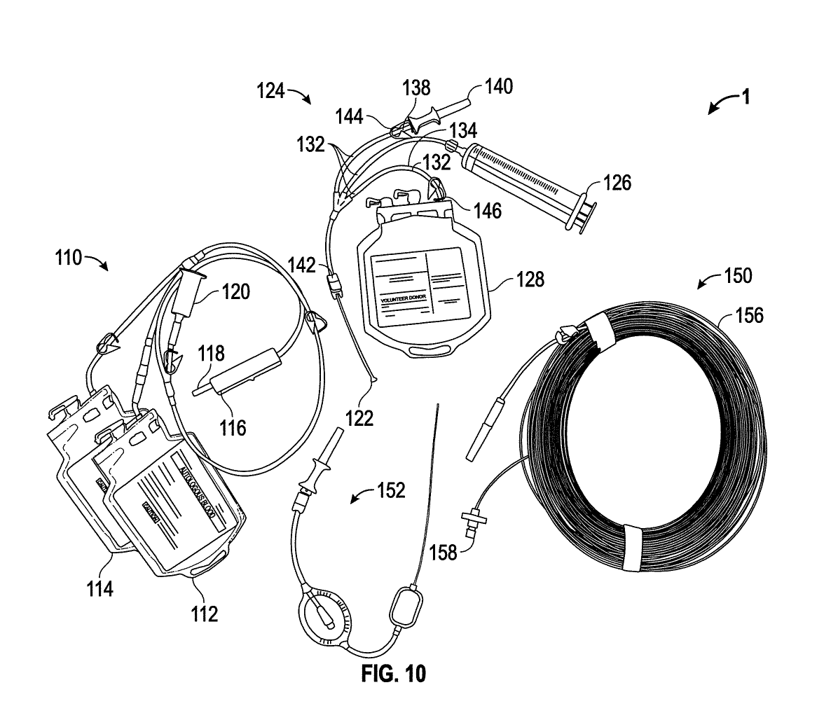

In operation, a patient who could benefit from autologous serum eye drops sees a doctor, nurse or other phlebotomist, who uses an exemplary autologous blood products collection kit 1, illustrated in FIG. 10. First, the medical specialist sets up the blood collection set 110, removes the needle guard 118 and inserts the needle 116 into a vein of the patient to draw blood. Some of the blood may be collected in sample tubes in a vacutainer adapter 120, and a portion of the blood is collected in the collection bag 112. In exemplary embodiments, the volume of blood collected in the collection bag 112 will be about 150 mL. The blood is allowed to clot in the collection bag 112, and then the blood may be spun in a centrifuge to separate the serum from the plasma, and the serum is expressed off through the tubing into the serum container or satellite bag 114.

Next, a laboratory technician uses the mixing system 130 including the branched dilution tube network 132 to direct the serum from the serum bag 114 of the blood collection set 110 through the serum input line 122 of the serum dilution set 124. The technician then directs the desired ratio of serum mixed with balanced saline solution using the mixing system 130. More particular, the technician directs balanced saline solution from a saline source through balanced saline solution line 138 and the serum through the serum input line 122 and uses the serum measurement device 126, e.g., an integral syringe, to draw measured amounts of serum and balanced saline solution.

The technician can regulate the flow of serum through the serum input line 122 by using first clamp 142. Similarly, the technician can use the second clamp 144 to control the flow of balanced saline solution through balanced saline solution line 138. The technician can use the third clamp 146 to control the flow of the serum-saline solution into the serum dilution bag 128. Once the serum-saline solution is diluted to the desired ratio in the serum dilution bag 128, the technician can use the aliquot set 150 dispense aliquots of solution for distribution and use as autologous serum eye drops.

First, the technician directs the diluted serum-saline solution through the materials filter 152 at the proximal end of the aliquot tubing 156 to remove any remaining clotted material from the serum-saline solution. Next, the diluted serum-saline solution is directed through the sterilization filter 154 to sterilize the diluted serum-saline solution. The now sterile final serum-saline solution is then directed into the approximately 1,000-inch long aliquot tubing 156. The technician may utilize an air vent 158 to allow any air in the aliquot tubing 156 to exit the tubing as the final serum-saline solution flows in and fills up the tube.

Once the aliquot tubing 156 is filled with the final serum-saline solution, the technician seals the individual short segments 162 of tubing to create segmented aliquots of final serum-saline solution. The technician can then separate the individual segmented aliquots 162 and, if desired, arrange them in a bundle 164 for transport or storage. If long-term storage is necessary, the technician may flash freeze the aliquots 162 to maintain their freshness and sterility. The technician can then ship the aliquots to a doctor or directly to a patient for use as autologous eye drops. Finally, the doctor or patient clips a corner 166 of the aliquot 162 and administers the drops to the eye through opening 168.

Advantageously, the above-described systems and methods allow for the controlled and sterile production of final diluted serum solution for use as autologous serum eye drops. The systems and methods can be used in a cGMP regulated laboratory environment which lends itself well to mass production.

Thus, it is seen that systems, methods and kits for production of autologus serum eye drops are provided. It should be understood that any of the foregoing configurations and specialized components or chemical compounds may be interchangeably used with any of the systems of the preceding embodiments. Although illustrative embodiments of the present invention are described hereinabove, it will be evident to one skilled in the art that various changes and modifications may be made therein without departing from the invention. It is intended in the appended claims to cover all such changes and modifications that fall within the true spirit and scope of the invention.

What is claimed is:

1. A system of making autologous blood products, comprising:
a blood collection assembly including a collection container and a serum container;
a dilution assembly fluidly connected to the blood collection assembly, the dilution assembly including a serum measurement device, a serum dilution bag, and a mixing system enabling serum to be mixed with balanced saline solution in a defined dilution ratio; and
an aliquot assembly fluidly connected to the dilution assembly, the aliquot assembly including at least one filter and aliquot tubing having periodic segment length markings.

2. The system of claim 1 further comprising a sealing device to seal each segment of aliquot tubing.

3. The system of claim 1 wherein the blood collection assembly further includes a needle and needle guard.

4. The system of claim 1 wherein the serum container is a 150 mL bag.

5. The system of claim 1 wherein the collection container is a 150 mL bag.

6. The system of claim 1 wherein the mixing device is a syringe, and the mixing system comprises:
a branched dilution tube including a balanced saline solution input line;
a serum container input line;
a first clamp to control flow of serum;
a second clamp to control flow of balanced saline solution; and
a third clamp to control flow of serum-saline solution into the serum dilution bag.

7. The system of claim 1 wherein the at least one filter comprises a materials filter and a sterilization filter.

8. The system of claim 7 wherein the materials filter is a 40 micron pre-filter and the sterilization filter is a 0.2 micron filter.

9. The system of claim 1 wherein each segment length marking contains a unique identifier.

10. The system of claim 1 wherein the aliquot tubing defines at least one air vent allowing air in the tubing to exit as the tubing is filled with serum-saline solution.

11. A method of making autologous blood products, comprising:
collecting blood from a patient and allowing the blood to clot;
separating blood serum and collecting the serum in a container;
mixing the serum with balanced saline solution in a defined dilution ratio;
filtering the serum-saline solution;
directing the serum-saline solution into aliquot tubing; and
producing a plurality of aliquots of serum-saline solution by sealing the aliquot tubing at periodic length segments.

12. The method of claim 11 further comprising freezing each aliquot of serum-saline solution.

13. The method of claim 11 wherein the filtering step comprises filtering of materials and sterilization filtering.

14. The method of claim 11 further comprising using beads to clot the collected blood.

15. The method of claim 11 further comprising marking each periodic length segment with a unique identifier.

16. An autologous blood products production kit, comprising:
a blood collection set including a collection container and a serum container;
a serum dilution set fluidly connected to the blood collection set, the serum dilution set including a serum measurement device, a serum dilution bag, and a mixing system enabling serum to be mixed with balanced saline solution in a defined dilution ratio; and
an aliquot set fluidly connected to the serum dilution set, the aliquot set including a materials filter, a sterilization filter, and aliquot tubing having periodic segment length markings.

17. The kit of claim 16, wherein the mixing system comprises:
a branched dilution tube including a balanced saline solution input line;
a serum container input line;
a first clamp to control flow of serum;
a second clamp to control flow of balanced saline solution; and
a third clamp to control flow of serum-saline solution into the serum dilution bag

18. The kit of claim 16 further comprising a sealing device to seal each segment of aliquot tubing.

19. The kit of claim 16 wherein the blood collection assembly further includes a needle and needle guard.

20. The kit of claim 16 wherein each segment length marking contains a unique identifier.
